(19) United States
(12) Patent Application Publication
(10) Pub. No.: US 2011/0231956 A1
(43) Pub. Date: Sep. 22, 2011

(54) Environmentally Adjusted Transgenic Plants

(75) Inventors: Villoo Morawala-Patell, Bangalore (IN); Mahesh Venkataramaiah, Bangalore (IN); Suhas Nimbalkar, Bangalore (IN); Manjula Ramakrishna, Bangalore (IN); Suresh Sadasivam, Bangalore (IN)

(73) Assignee: Avesthagen Limited, Bangalore (IN)

(21) Appl. No.: 13/054,398

(22) PCT Filed: Jul. 7, 2009
(86) PCT No.: PCT/IB2009/006225

§ 371 Date: Jan. 14, 2011

(30) Foreign Application Priority Data
Jul. 14, 2008 (IN) 1699/CHE/2008

(51) Int. Cl. A01H 5/00; C12N 15/82

(52) U.S. Cl. 800/287; 800/278; 800/298

(57) ABSTRACT

The present invention relates to a novel transgenic plant having tolerance to salt stress. The plant is transformed with a recombinant nucleic acid encoding glutamic acid decarboxylase isolated from Oryza sativa. Still further it also relates to a method of producing the transgenic plants that are salt tolerant.

### FIELD OF THE INVENTION

The present invention relates to transgenic plants, which are salt tolerant. In particular, the present invention relates to transgenic plants that express glutamate decarboxylase, and to methods for preparing such transgenic plants.

### BACKGROUND OF THE INVENTION

Salinity stress negatively impacts agricultural yield throughout the world affecting production whether it is for subsistence or economic gain. The plant response to salinity consists of numerous processes that must function in coordination to alleviate both cellular hyperosmolarity and ion disequilibrium. In addition, crop plants must be capable of satisfactory biomass production in a saline environment.

In the present invention methods and materials for making plants having an enhanced ability to withstand environmental stress and having desirable morphological and/or agronomic characteristics or the like, are provided through plant genetic engineering. More particularly, the invention relates to genetic transformation of plants with genes that enhance a plant's ability to synthesis glutamate decarboxylase enzyme, which catalyzes the conversion of glutamic acid to GABA thereby enhancing the plant's ability to withstand stress or imparts other desirable characteristics.

As a background to the invention, the enzyme GAD (glutamic acid decarboxylase) has been shown to catalyze the formation of gamma amino butyric acid (GABA) from glutamate (Glu), and several plant GAD genes have been cloned. The rapid accumulation of GABA in plant cells after exposure to stress has been well documented. The production of GABA by decarboxylation of glutamate facilitated by the enzyme GAD is proposed to be the major source through which GABA accumulates in plants after stress. However GABA is also biosynthesized by other metabolic pathways like the one associated with the catabolism of polyamines or through a part of GABA shunt by the reversible GABA amino transferase reaction. Experiments with soybean cotyledons or Asparagus cell suspension culture suggests that formation of GABA by the metabolism of glutamate is a normal phenomenon and that biosynthesis of GABA is not a response to stress under the conditions studied.

However GABA has also been shown to rapidly accumulate in plants subjected to mechanical stimulation, variation in temperatures like cold or heat shock conditions. In view of this background, it is seen that significant effort has been devoted to studying GABA synthesis and GAD enzyme activity in plants; however, a direct role for GABA in plants towards imparting salinity tolerance has not heretofore been demonstrated. The present invention is a significant advance in this field.

### PRIOR ART

### Mechanisms of Salt Tolerance

The early discovery by biochemists that enzymes of halophytes (plants adapted to saline habitats) are no more tolerant of high concentrations of NaCl than are those of non-halophytes (also called glycophytes, or plants adapted to sweet water) underlies all mechanisms of salt tolerance (Munns 2002). For example, in vitro activities of enzymes extracted from the halophytes Atriplex spongeosa or Suaeda maritima were just as sensitive to NaCl as were those extracted from beans or peas (Greenway & Osmond 1972; Flowers et al. 1977). Even enzymes from the pink salt-lake alga Dunaliella parva, which can grow at salinities 10-fold higher than those of seawater, are as sensitive to NaCl as those of the most sensitive glycophytes (reviewed by Munns et al. 1983). Generally, Na⁺ starts to inhibit most enzymes at a concentration above 100 mM. The concentration at which Cl⁻ becomes toxic is even less well defined, but is probably in the same range as that for Na⁺. Even K⁺ may inhibit enzymes at concentrations of 100-200 mM (Greenway & Osmond 1972).

Mechanisms for salt tolerance are therefore of two main types: those minimizing the entry of salt into the plant, and those minimizing the concentration of salt in the cytoplasm. Halophytes have both types of mechanisms; they ‘exclude’ salt well, but effectively compartmentalize in vacuoles the salt that inevitably gets in. This allows them to grow for long periods of time in saline soil. Some glycophytes also exclude the salt well, but are unable to compartmentalize the residual salt taken up as effectively as do halophytes. Most glycophytes have a poor ability to exclude salt, and it concentrates to toxic levels in the transpiring leaves

High salinity conditions result in hyperosmotic damage to most plants, and elevated Na⁺ concentration disrupts cellular processes by interfering with vital Na⁺-sensitive enzymes and by affecting essential ion transport. It is thought that Na⁺ uptake occurs via multiple Na⁺ permeable channels/transporters under saline conditions and that ion toxicity is triggered when the cytoplasmic Na⁺ concentration reached some threshold level (Volkamar et al., 1999; Hasegawa et al., 2000). To genetically enhance the salt-tolerance of plants, a rational strategic approach should be followed in order to endow resistance against the above-mentioned stresses. Most plants synthesize and accumulate osmolytes, so called compatible solutes, as a response to drought or high salinity conditions. These compatible solutes are neutral under physiological pH, have low molecular mass, high solubility in water, and are nontoxic to the organisms even when accumulated at high concentrations in the cytosol. Some transgenic plants into which genes for bio-synthesis of osmolytes were introduced, such as mannitol (Tarczynski et al., 1993), ononitol (Sheveleva et al., 1997), trehalose (Holmstriim et al., 1996; Romero et al., 1997), proline (Kishor et al., 1995), betaine (Lilius et al., 1996; Hyashi et al., 1997; Sakamoto et al., 1998), or fructan (Pilon-Smits et al., 1995), ectoine (1,4,5,6-tetrahydro-2-methyl-4-pyrimidinecarboxylic acid), a compatible solute in the halophile Halomonas elongata (Yoshida 2002), myoinositol (Das-Chatterjee et al., 2006) showed improved hyperosmotic tolerance. As another strategy, it was reported that over-expression of the Arabidopsis thaliana DREBIA gene, which encodes a transcription factor that regulates the expression of stress tolerance genes, resulted in improved tolerance of the transgenic plants to drought, salinity, and freezing (Kasuga et al., 1999). In general, molecular improvement of tolerance to Na⁺ toxicity is difficult. There have been only few reports of improved tolerance through over-expression of a vacuolar Na⁺/H⁺ antiporter gene (NHXI) or vacuolar proton pump gene (AVPZ) in A. thaliana (Apse et al., 1999). We currently demonstrate the enhanced salt stress tolerance of plant cells by introducing the Oryza sativa GAD gene, which encodes a glutamate decarboxylase enzyme.

### GABA Shunt

Gamma-Amino butyric acid (GABA) is a four-carbon non-protein amino acid conserved from bacteria to plants and vertebrates. GABA is a significant component of the free amino acid pool. GABA has an amino group on the gamma-carbon rather than on the alpha-carbon, and exists in an unbound form. It is highly soluble in water: structurally it is a flexible molecule that can assume several conformations in solution, including a cyclic structure that is similar to proline 1. GABA is zwitterionic (carries both a positive and negative charge) at physiological pH values (pK values of 4.03 and 10.56).

It was discovered in plants more than half a century ago, but interest in GABA shifted to animals when it was revealed that GABA occurs at high levels in the brain, playing a major role in neurotransmission. Thereafter, research on GABA in vertebrates focused mainly on its role as a signaling molecule, particularly in neurotransmission. In plants and in animals, GABA is mainly metabolized via a short pathway composed of three enzymes, called the GABA shunt because it bypasses two steps of the tricarboxylic acid (TCA) cycle. The pathway is composed of the cytosolic enzyme glutamate decarboxylase (GAD) and the mitochondrial enzymes GABA transaminase (GABA-T) and succinic semialdehyde dehydrogenase (SSADH). The regulation of this conserved metabolic pathway seems to have particular characteristics in plants.

The pathway that converts glutamate to succinate via GABA is called the GABA shunt. The first step of this shunt is the direct and irreversible α-decarboxylation of glutamate by glutamate decarboxylase (GAD, EC 4.1.1.15). In vitro GAD activity has been characterized in crude extracts from many plant species and tissues (Brown & Shelp, 1989). GAD is specific for L-glutamate, pyridoxal 5′-phosphate-dependent, inhibited by reagents known to react with sulfhydryl groups, possesses a calmodulin-binding domain, and exhibits a sharp acidic pH optimum of ˜5.8. GAD genes from Petunia (Baum et al., 1993), tomato (Gallego et al., 1995), tobacco (Yu & Oh, 1998) and Arabidopsis (Zik et al., 1998) have been identified. The second enzyme involved in the GABA shunt, GABA transaminase (GABA-T; EC 2.6.1.19), catalyzes the reversible conversion of GABA to succinic semialdehyde using either pyruvate or α-ketoglutarate as amino acceptors. In crude extracts, in vitro GABA-T activity appears to prefer pyruvate to α-ketoglutarate. However, distinct pyruvate-dependent and α-ketoglutarate-dependent activities are present in crude extracts of tobacco leaf, and these can be separated from each other by ion exchange chromatography (Van Cauwenberghe & Shelp). Both activities exhibit a broad pH optimum from 8 to 10. The Michaelis constants (Km) of a pyruvate-specific mitochondrial GABA-T from tobacco, purified ˜1000-fold, are 1.2 mM for GABA and 0.24 mM for pyruvate (Van Cauwenberghe & Shelp).

The last step of the GABA shunt is catalyzed by succinic semialdehyde dehydrogenase (SSADH; EC 1.2.1.16), irreversibly oxidizing succinic semialdehyde to succinate. The partially purified plant enzyme has an alkaline pH optimum of ˜9; activity is up to 20-times greater with NAD than with NADP (Shelp et al., 1995).

Indeed, interest in the GABA shunt in plants emerged mainly from experimental observations that GABA is largely and rapidly produced in response to biotic and abiotic stresses. The GABA shunt has since been associated with various physiological responses, including the regulation of cytosolic pH, carbon fluxes into the TCA cycle, nitrogen metabolism, deterrence of insects, protection against oxidative stress, osmoregulation and signaling.

### Protection Against Oxidative Stress

In Arabidopsis, mutants disrupted in succinic semialdehyde dehydrogenase are more sensitive to environmental stress because they are unable to scavenge H₂O₂ (Bouche et al., 2003). The last step of the GABA shunt can provide both succinate and NADH to the respiratory chain. It was therefore hypothesized that the degradation of GABA could limit the accumulation of reactive oxygen intermediates under oxidative stress conditions that inhibit certain enzymes of the TCA cycle. In yeast, mutants knocked out in GABA-shunt genes seem to be more sensitive to H₂O₂ (Coleman et al., 2001).

The work of Coleman et al., 2001, provides insight into the intracellular involvement of GAD in oxidative stress tolerance. Increasing the gene dosage of the S. cerevisiae GAD1 locus produced an increased tolerance to two different oxidative agents, diamide and H₂O₂. This increased tolerance was strictly dependent on the presence of the intact glutamate catabolic pathway leading to the production of succinate from glutamate. Genetic elimination of either enzymatic reaction downstream from glutamate decarboxylase rendered cells hypersensitive to oxidants.

### Synthesis/Overexpression of Compatible Solutes

The cellular response of salt-tolerant organisms to both long- and short-term salinity stresses includes the synthesis and accumulation of a class of osmoprotective compounds known as compatible solutes. These relatively small organic molecules are not toxic to metabolism and include proline, glycinebetaine, polyols, sugar alcohols, and soluble sugars. These osmolytes stabilize proteins and cellular structures and can increase the osmotic pressure of the cell (Yancey et al., 1982). This response is homeostatic for cell water status, which is perturbed in the face of soil solutions containing higher amounts of NaCl and the consequent loss of water from the cell. Glycinebetaine and trehalose act as stabilizers of quartenary structure of proteins and highly ordered states of membranes. Mannitol serves as a free radical scavenger. It also stabilizes sub cellular structures (membranes and proteins), and buffers cellular redox potential under stress. Hence these organic osmolytes are also known as osmoprotectants (Bohnert and Jensen, 1996; Chen and Murata, 2000).

### Compatible Osmolyte

AtProT2 can be induced by water stress, and AtProT2 and LeProT1 transport GABA as well as other stress-related compounds, such as proline and glycine betaine (Breitkreuz, et al. 1999; Schwacke, et al. 1999; Fischer, et al. 1998). These findings indicate that GABA might have a role as a compatible osmolyte (Yancey 1994). All three compounds are zwitterionic at neutral pH, are highly soluble in water, can accumulate to low mM concentrations, and apparently contribute no toxic effects to the cell. At high concentrations (25-200 mM), GABA stabilizes and protects isolated thylakoids against freezing damage in the presence of salt, exceeding the cryoprotective properties of proline. In addition, GABA possesses in vitro hydroxyl-radical-scavenging activity, exceeding that of proline and glycine betaine at the same concentrations (16 mM) (Smirnoff & Cumbes 1989). GABA might be synthesized from γ-amino-butyraldehyde (a product of the polyamine catabolic pathway) by the chloroplast-localized betaine aldehyde dehydrogenase, which is involved in glycine betaine synthesis (Trossat et al., 1997), but the relative fluxes via polyamines versus glutamate decarboxylation are unknown.

### OBJECTS OF THE INVENTION

The present invention relates to a method of increasing salt tolerance in plants (monocotyledons and dicotyledons) via Agrobacterium-mediated transformation with a glutamate decarboxylase gene. Further more the present invention relates to a method of plant modification to express genes, related to salt tolerance and to the plants produced using this method.

A method employing the glutamate decarboxylase gene from rice to increase the salt tolerance of plants has been demonstrated. Earlier attempts have been made in this direction using osmolytes like mannitol, ononitol, trehalose, proline, betaine, or fructan, ectoine, myoinositol.

Abiotic stress is a complex environmental constraint limiting crop production. A bioengineering stress-signaling pathway to produce stress-tolerant crops is one of the major goals of agricultural research. Osmotic adjustment is an effective component of such manipulations and accumulation of osmoprotectants (compatible solutes) is a common response observed in plant systems (Penna 2003). Other mechanisms by which compatible solutes protect plants from stress include detoxifying radical oxygen species and stabilizing the quaternary structures of proteins to maintain their function.

Given the complexity of the physiology and the genetics of salt tolerance, it has been a difficult task to generate salt-tolerant crops. There has been only limited success in this direction in the mid-1990s (Flowers and Yeo, 1995) and there has been little progress since then. A variety of approaches have been advocated, including conventional breeding, wide crossing, the use of physiological traits and, more recently, marker-assisted selection and the use of transgenic plants. None of these approaches could be said to offer a universal solution. Conventional breeding programs have rarely delivered enhanced salt tolerance (Flowers and Yeo, 1995), while wide crossing generally reduces yield to unacceptably low levels (Yeo and Flowers, 1981). There has been success using physiological criteria as the basis of selection of rice (Dedolph and Hettel, 1997) and such an approach has recently been advocated for wheat (Munns et al., 2002). A recent analysis has shown that while it is possible to produce a wide range of transgenic plants where some aspect of a trait relating to salt tolerance was altered, none has been tested in the field and few claims for success meet even minimal criteria required to demonstrate enhanced tolerance (Flowers, 2004).

No attempt has been made so far to use the genes involved in GABA shunt pathway, specifically glutamate decarboxylase from rice to increase the salt tolerance of plants. Previous attempts directed at two glutamate decarboxylase genes from rice OsGAD1 and OsGAD2, which were introduced simultaneously into rice calli via Agrobacterium to establish transgenic cell lines. Regenerated rice plants had aberrant phenotypes such as dwarfism, etiolated leaves, and sterility (Akama & Takaiwa, 2007).

### SEQUENCE LISTING

SEQ ID 1 shows the nucleic acid sequence of Oryza sativa glutamate decarboxylase gene. The start and stop codons are in italic.

SEQ ID 2 shows amino acid sequence of Oryza sativa glutamate decarboxylase gene. The asterisk denotes the stop codon.

### BRIEF DESCRIPTION OF ACCOMPANYING FIGURES

FIG. 1 shows the plant transformation vector harboring the glutamate decarboxylase encoding DNA sequence.

FIG. 2 shows the different stages in the transformation of tobacco leaves with GAD gene through Agrobacterium mediated gene transfer

FIG. 3 shows the PCR confirmation of the transformed and regenerated T0 seedlings of tobacco with GAD gene with different combination of primers—a) HygR-gene forward and reverse; b) Gene specific forward and reverse primer and c) Gene forward and Nos reverse primer

FIG. 4 shows the confirmation of the expression of the introduced gene (GAD) in T0 seedlings of tobacco with GAD gene analyzed using RT-PCR on cDNA as template with GAD gene specific forward and reverse primers.

FIG. 5 shows the better performance of T1 GAD transgenic tobacco seedlings (D1A, E2 and H1) under salt stress conditions (200 mM NaCl) grown on agar media in the light room.

FIG. 6 shows the better performance of T1 GAD transgenic tobacco seedlings (E2 and H1) under salt stress conditions (300 mM NaCl) grown on hydroponics culture in the green house.

FIG. 7 shows comparison of plant height between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 8 shows comparison of internodal distance between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 9 shows comparison of number of leaves between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 10 shows comparison of stem girth or thickness between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 11 shows comparison of leaf area between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 12 shows comparison of total biomass between T1 Seedlings from GAD transgenics (D1A, E2 and H1), which were positive for Hygromycin and the wild type seedlings when grown in pots in the green house with different levels of salt stress (0, 200 & 300 mM NaCl).

FIG. 13 shows comparison of total grain yield between T1 Seedlings from GAD transgenics (D1A, E2 and H1), which were positive for Hygromycin and the wild type seedlings when grown in pots in the green house with different levels of salt stress (0, 200 & 300 mM NaCl).

### DETAILED DESCRIPTION OF THE INVENTION

The following detailed description of the invention is provided to aid those skilled in the art in practicing the present invention. Even so, the following detailed description of the invention should not be construed to unduly limit the present invention as modifications and variations in the embodiments discussed herein may be made by those of ordinary skill in the art without departing from the spirit or scope of the present invention.

This invention relates to a purified and isolated DNA sequence having characteristics of glutamate decarboxylase.

According to the present invention, the purified and isolated DNA sequence usually consists of a glutamate decarboxylase nucleotide sequence or a fragment thereof.

Included in the present invention are as well complementary sequences of the above-mentioned sequences or fragment, which can be produced by any means.

Encompassed by this present invention variants of the above mentioned sequences, that is nucleotide sequences that vary from the reference sequence by conservative nucleotide substitutions, whereby one or more nucleotides are substituted by another with same characteristics.

According to the present invention, the above mentioned nucleotide sequences could be located at both the 5′ and the 3′ ends of the sequence containing the promoter and the gene of interest in the expression vector.

Included in the present invention are the use of above mentioned sequences in increasing the salt tolerance of the plants produced thereof. “salt tolerance” means that after introduction of DNA sequence under suitable conditions into a host plant, the sequence is capable of enhancing the plants capacity to withstand high concentrations of salts in the growing environments in the plants as compared to control plants where the plants are not transfected with the said DNA sequence.

The following definitions are used in order to help in understanding the invention.

“Chromosome” is organized structure of DNA and proteins found inside the cell.

“Chromatin” is the complex of DNA and protein, found inside the nuclei of eukaryotic cells, which makes up the chromosome.

“DNA” or Deoxyribonucleic Acid, contain genetic informations. It is made up of different nucleotides.

A “gene” is a deoxyribonucleotide (DNA) sequence coding for a given mature protein. “gene” shall not include untranslated flanking regions such as RNA transcription initiation signals, polyadenylation addition sites, promoters or enhancers.

“Promoter” is a nucleic acid sequence that controls expression of a gene.

“Enhancer” refers to the sequence of gene that acts to initiate the transcription of the gene independent of the position or orientation of the gene.

The definition of “vector” herein refers to a DNA molecule into which foreign fragments of DNA may be inserted. Vectors, usually derived from plasmids, functions like a “molecular carrier”, which will carry fragments of DNA into a host cell.

“Plasmid” are small circles of DNA found in bacteria and some other organisms. Plasmids can replicate independently of the host cell chromosome.

“Transcription” refers the synthesis of RNA from a DNA template.

“Translation” means the synthesis of a polypeptide from messenger RNA.

“Orientation” refers to the order of nucleotides in the DNA sequence.

“Gene amplification” refers to the repeated replication of a certain gene without proportional increase in the copy number of other genes.

“Transformation” means the introduction of a foreign genetic material (DNA) into plant cells by any means of transfer. Different method of transformation includes bombardment with gene gun (biolistic), electroporation, Agrobacterium mediated transformation etc.

“Transformed plant” refers to the plant in which the foreign DNA has been introduced into the said plant. This DNA will be a part of the host chromosome.

“Stable gene expression” means preparation of stable transformed plant that permanently express the gene of interest depends on the stable integration of plasmid into the host chromosome.

### Example 1

### Isolation and Purification of GAD Gene Nucleotide Sequence from Rice and Construction of Plant Transformation Vector

The GAD gene is cloned downstream of a 35S cauliflower mosaic virus promoter and terminated with a NOS terminator, all operably linked.

### Plant Materials

Oryza sativa (cv Rasi) was used for preparation of nucleic acids. After germination of the seeds, they were grown in hydroponic solution in a culture room. The seedlings were treated with 150 mM NaCl for 7-16 h.

### RNA Extraction and EST Library Construction

The RNA was extracted from the whole seedlings. An EST library of the salt stressed RASI cDNA was constructed. An EST showing identity to glutamate decarboxylase was identified from the EST library.

### Identification and Isolation of Genes in the GABA Shunt

GABA accumulates in higher plants following the onset of a variety of stresses such as acidification, oxygen deficiency, low temperature, heat shock, mechanical stimulation, pathogen attack, drought and salt stress. Glutamate decarboxylase, the gene in the GABA shunt has been isolated from the salt stressed library of O. sativa.

### Cloning of Glutamate Decarboxylase Gene

The Glutamate decarboxylase gene has been cloned into a cloning vector and also into plant transformation vectors (biolistic and binary) under a constitutive promoter. The cDNA encoding the complete coding sequence of glutamate decarboxylase gene was amplified from the indica rice (cv. RASI) cDNA using the following pairs of primers tagged with BglII and EcoRI restriction enzyme sites (underlined nucleotide sequences)

Forward: 5′-GCGGATCCATGGTGCTCTCCAAGGCCGTCTC-3′
Reverse: 5′-GCGAATTCCTAGCAGACGCCGTTGGTCCTCTTG-3′

Using the following PCR conditions 94° C. for 1 min; 94° C. for 30 sec; 75° C. for 3 min (cycled for five times); 94° C. for 30 sec; 68° C. for 3 min (cycled for 30 times) with a final extension of 68° C. for 7 min.

The amplified cDNA consists of 1479 base pairs of nucleotides and encodes for a mature glutamate decarboxylase enzyme.

The amplified fragment was cloned into pGEMT easy vector. The gene was restriction digested at BamHI and EcoRI sites and ligated into a biolistic vector pV1. This biolistic vector was excised at BglII and EcoRI restriction sites (BglII and BamHI enzymes generate compatible ends) to confirm the presence of the gene. The gene was also confirmed by sequencing. The resultant vector (pV1-GAD) has the GAD gene (1.479 kb) driven by 35S Cauliflower Mosaic virus (35S CaMV) promoter and NOS terminator along with the ampicillin resistance gene as a selectable marker.

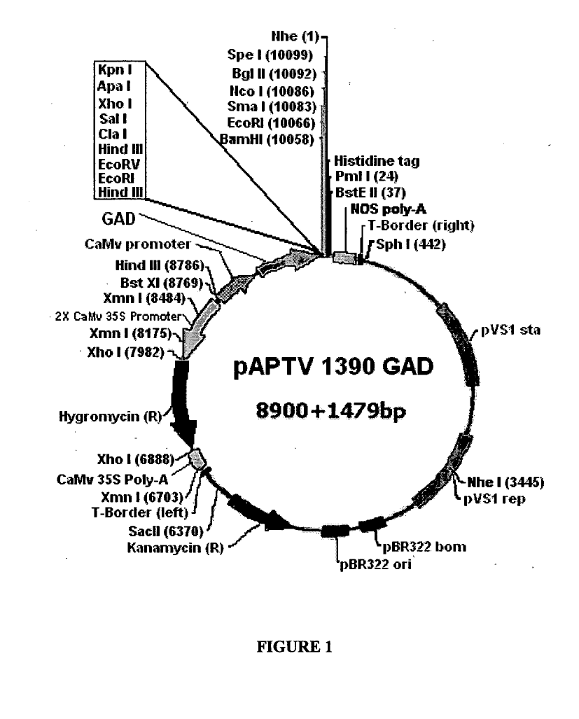

The gene cassette, GAD gene driven by the 35S CaMV promoter and terminated by the NOS terminator from pV1-GD was restriction digested at HindIII and BamHI sites. This gene cassette was ligated into pCAMBIA 1390 pNG15 which was restriction digested at HindIII and BamHI sites. The resultant vector (pAPTV 1390-GAD) has the GAD gene (1.479 kb) driven by 35S cauliflower mosaic virus (35S CaMV) promoter and terminated by NOS terminator along with the nptII (Kanamycin resistance) gene and hph gene (Hygromycin resistance) as selectable markers (FIG. 1).

### Example 2

Generating Plants with an Altered GAD Gene

### Plant Transformations

The Glutamate decarboxylase gene has been transformed via Agrobacterium into tobacco (model plant) to arrive at the proof of concept for the identified gene.

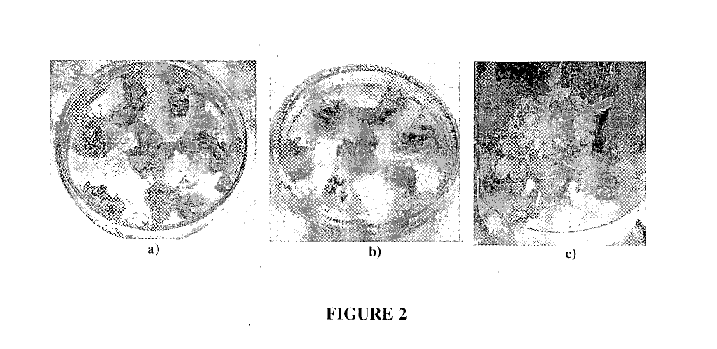

Detailed steps involved in Agrobacterium mediated transformation of tobacco leaf explants with a binary vector harboring GAD gene:
1. The positive colony of Agrobacterium was inoculated in to LB broth with 50 mg/L Kanamycin (Kan) and 10 mg/L of Rifamicin (Rif) as vector backbone consists of Kan and Rif resistance gene, which also functions as double selection at one shot.
2. Then the broth was incubated at 28° C. on a shaker.
3. The overnight grown colony was inoculated into 50 mL LB broth with 50 mg/L Kan and 10 mg/L of Rif in the morning and incubated at 28° C. for 3-4 hours and the OD was checked at 600 nm and continued to grow till the OD was between 0.6-1.
4. Once the broth reached required OD the broth was centrifuged at 5000 rpm for 5 min.
5. The supernatant was discarded and the cell pellet was dissolved in Murashige & Skooge (MS) liquid medium (Agro-MS broth).
6. The tobacco leaves were cut in to small square pieces which served as explants with out taking the midrib and care was taken to injure leaf at all four sides with out injuring much at the center part of the inoculants.
7. These leaf samples were placed in MS Plain media for two days in a BOD incubator. After two days of inoculation these leaf samples were infected with transformed Agrobacterium cells, which are now in Agro-MS broth.
8. The leaf explants were placed in this Agro-MS broth for 30 min and then placed them on co-cultivation media, which consist of MS+1 mg/L 6-Benzyl amino purine hydrochloride (BAP)+0.2 mg/L Naphthalene acetic acid (NAA)+250 mg/L Cefotaxime for two days (FIG. 2a)
9. After co-cultivation the explants were kept in first selection medium which consist of MS+1 mg/L BAP+0.2 mg/L NAA+40 mg Hyg+250 mg/L Cefotaxime for 15 days and as the callus started protruding these explants were again sub cultured on to first selection media for callus to mature enough (FIG. 2b)
10. Once the callus was found to be matured these callus were inoculated on to second selection medium which consist of MS+1 mg/L BAP+0.2 mg/L NAA+50 mg Hyg+250 mg/L Cefotaxime. As the concentration of Hygromycin is increased the escapes from first selection get suppressed and only the transformed callus starts surviving on this media.
11. Subsequent sub-cultures on this second selection media were done once in ten days.
12. By this time the plantlets started protruding from the callus. The plantlets from second selection were taken and placed on to rooting media, which consist of ½ MS+0.2 mg/L Indole-3-butyric acid (IBA). Here the plantlets started protruding roots by 12-15 days. Once the mature roots were formed the plants were subcultured on to rooting media along with 20 mg/L of Hygromycin, as escapes can be identified at this stage also (FIG. 2c).
13. Plants at this stage were subjected to acclimatization where the caps of bottles were kept open for two days so that plants get adjusted to its growth room environment. Later plants from agar medium were removed and placed on ¼ MS liquid medium for two days. These plants were further transferred on to vermiculate and watered every day for one week.
14. Depending upon the condition of the plants suitable plants were transferred to green house.
15. Before sending plants to green house during acclimatization period old leaves from the plants were collected.
16. DNA from respective leaf samples was extracted and PCR with gene specific primers and selection marker gene i.e. Hygromycin primers were performed. PCR confirmed positive plants were further transferred to green house.
   Confirmation of Plants with Introduced GAD Gene

### Genomic DNA Extraction of GAD Tobacco Transgenic Lines

Leaf samples of transgenic GAD tobacco plant were collected and genomic DNA was extracted.

### Procedure for Genomic DNA Extraction:

Around 1 gm of leaf was collected from each plant.
The samples were ground using liquid nitrogen in a pestle and mortar.
1 ml of extraction buffer Extraction buffer (0.2M Tris Cl pH-8.0; 2 M NaCl; 0.05 M EDTA; 2% CTAB) was added to the sample and spun at 13000 rpm for 10 min
Supernatant was collected. RNase [3 μl (1 mg/mL) for 1 ml] was added and incubated at 37° C. for ½ an hour.
Equal volumes of chloroform-isoamyl alcohol was then added and spun at 13000 rpm for 10 min.
Supernatant was collected in fresh tubes and equal volumes of chilled Isopropanol was added and spun at 13000 rpm for 10 min.
The pellet was washed with 70% alcohol and pellet was dried and dissolved in 30 μl warm autoclaved water.
1 μl of DNA was loaded and checked on gel.
   The Transgenic Plants were Confirmed by PCR with Different Combination of Primers:
   1. PCR with Hygromycin Forward (Hyg F) & Hygromycin Reverse (Hyg R) Primers:

| Reagent | Stock | Volume |
|---|---|---|
| Template DNA | | 1 μl |
| Hyg F | 10 pM | 0.5 μl |
| Hyg R | 10 pM | 0.5 μl |
| dNTP's | 10 mM | 0.5 μl |
| Taq DNA polymerase | 3 U/μl | 0.3 μl |
| Taq buffer A | 10X | 3 μl |
| Milli Q water | | 24.2 μl |
| Total volume | | 30 μl |

PCR conditions: (Eppendorf Machine)

| Steps | Temperature | Time | Cycle |
|---|---|---|---|
| 1 | 94° C. | 3 mins | |
| 2 | 94° C. | 30 secs | |
| 3 | 50° C. | 50 secs | |
| 4 | 72° C. | 1 min | Go to step-2 30X |
| 5 | 72° C. | 10 mins | |
| 6 | 10° C. | ∞ | |

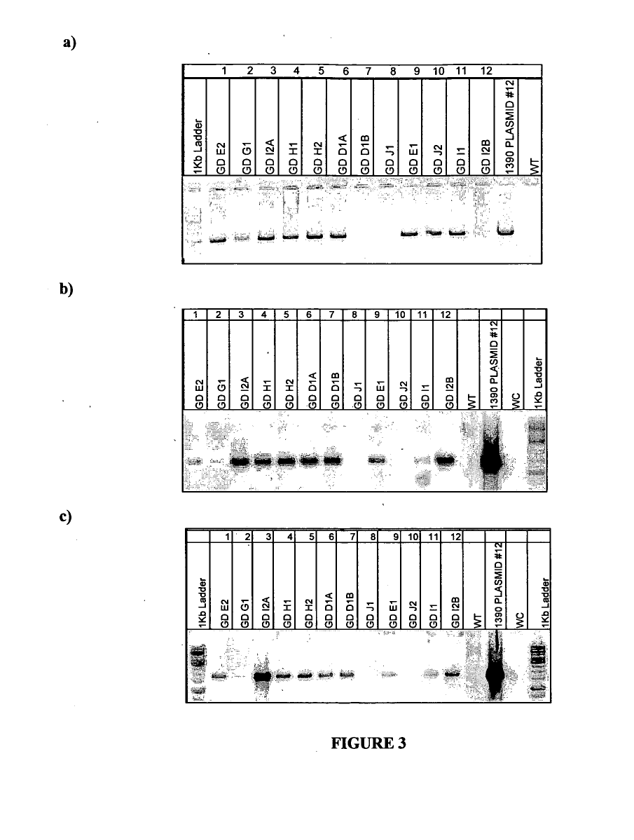

The amplified product was visualized on 0.8% agarose gel shown in FIG. 3a.

2. PCR with Gene Specific Primers GAD Forward (GD F) & GAD Reverse (GD R):

| Reagent | Stock | Volume |
|---|---|---|
| Template DNA | | 2 μl |
| GD F | 10 pM | 0.5 μl |
| GD R | 10 pM | 0.5 μl |
| dNTP's | 10 mM | 0.5 μl |
| Taq DNA polymerase | 3 U/μl | 0.3 μl |
| Taq buffer A | 10X | 2 μl |
| Milli Q water | | 14.2 μl |
| Total volume | | 20 μl |

### PCR Conditions: (Eppendorf Machine)

| Steps | Temperature | Time | Cycle |
|---|---|---|---|
| 1 | 94° C. | 3 mins | |
| 2 | 94° C. | 30 secs | |
| 3 | 69° C. | 50 secs | |
| 4 | 72° C. | 1.30 min | Go to step-2 35X |
| 5 | 72° C. | 10 mins | |
| 6 | 10° C. | ∞ | |

The amplified product was visualized on 0.8% agarose gel (FIG. 3b)

3. PCR with GD F & Nos MR:

| Reagent | Stock | Volume |
|---|---|---|
| Template DNA | | 2 μl |
| GD F | 10 pM | 0.5 μl |
| Nos MR | 10 pM | 0.5 μl |
| dNTP's | 10 mM | 0.5 μl |
| Taq DNA polymerase | 3 U/μl | 0.3 μl |
| Taq buffer A | 10X | 2 μl |
| Milli Q water | | 14.2 μl |
| Total volume | | 20 μl |

### PCR Conditions: (Eppendorf Machine)

| Steps | Temperature | Time | Cycle |
|---|---|---|---|
| 1 | 94° C. | 3 mins | |
| 2 | 94° C. | 30 secs | |
| 3 | 67° C. | 50 secs | |
| 4 | 72° C. | 2 min | Go to step-2 35X |
| 5 | 72° C. | 10 mins | |
| 6 | 10° C. | ∞ | |

The amplified product was visualized on 0.8% agarose gel shown in FIG. 3c.

Primer sequences used in different PCR reactions are listed below:

| | |
|---|---|
| Hyg F: | 5′-CTGAACTCACCGCGACGTCT-3′ |
| Hyg R: | 5′-CCACTATCGGCGAGTACTTC-3′ |
| GD F: | 5′-GCGGATCCATGGTGCTCTCCAAGGCCGTCTC-3′ |
| GD R: | 5′-GCGAATTCCTAGCAGACGCCGTTGGTCCTCTTG-3′ |
| NOS MR: | 5′-GATAATCATCGCAAGACCGGCAAC-3′ |

### Confirmation of Expression of the Introduced GAD Gene in the Transgenic Plants

The confirmation of the expression of the introduced GAD gene involved steps like RNA extraction, cDNA synthesis and Reverse Transcription PCR.

RNA of transgenic GAD tobacco plants along with the control plant (wild type) was isolated.

### Detailed Steps Involved in RNA Extraction:

1. 500 mg of leaf tissue was taken in prechilled mortar and ground in liquid nitrogen to fine powder.
2. The powder was transferred to a prechilled eppendorf tube using a chilled spatula.
3. 1 ml of Trizol solution (Invitrogen) was added to the homogenized sample. Mixed well and incubated at room temperature (RT) for 5 min.
4. 200 μl of chloroform was added to it and shaken vigorously for 15 seconds and incubated at room temperature for 5 mins.
5. The samples were centrifuged at 13000 rpm for 15 min at 4° C.
6. The upper aqueous phase was collected in a fresh tube (Approximately 60% i.e. 6000)
7. 500 μl of cold Isopropanol was added to the upper phase collected and incubated at RT for 10 min.
8. The samples were centrifuged at 13000 rpm for 15 min at 4° C.
9. The supernatant was decanted and the pellet washed with 500 μl of 70% alcohol (DEPC H₂O) and centrifuged at 10000 rpm for 5 minutes at 4° C.
10. The supernatant was decanted and the pellet dried for 15 min at RT.
11. The pellet was dissolved in 20 μl of DEPC treated H₂O in a heating water bath or dry bath set at 55° C.
12. 2 μl of the sample is loaded on the gel. Stored the sample at −80° C. till further use.
   Detailed Steps Involved in cDNA Synthesis:

cDNA synthesis of transgenic GAD tobacco plants along with the wild type was done.
1. The components were added in the order given below:
   Total RNA: 4 ul (1 ug)
   Oligo dT's: 0.5 ul
   0.1% DEPC/nuclease free water: 6.5 ul
   Total: 11 ul
2. The contents were heated at 70° C. for 5 min in a PCR machine and snap chilled in ice.
3. Meanwhile the next mixture was prepared by adding the following components in another tube:
   5× reaction buffer: 4 ul
   dNTP's (10 mM): 2 ul
   RNase inhibitor (20 U/ul): 0.5 ul
   0.1% DEPC/nuclease free water: 2 ul
   Total: 8.5 ul
4. This 8.5 ul mixture was added to the content in PCR tube, which was snap chilled and mixed by gentle tapping.
5. The contents were incubated in PCR tube at 37° C. for 5 minutes in a PCR machine.
6. 0.5 ul of the M-MuLV RT enzyme was added to the tube and continued the program set in the PCR machine (25° C. for 10 min; 37° C. for 60 min and 70° C. for 10 min).
7. Store the cDNA at −20° C. till further use in PCRs.

### Analysis of Expression of the Introduced GAD Gene in the Transgenic Tobacco Plants by RT-PCR

The cDNA samples from GAD transgenic tobacco and wild type plant were analyzed by PCR with Gene specific primers to check for the expression of the introduced GAD gene in tobacco:

PCR of cDNA with Gene Specific Primers:

| Reagent | Stock | Volume |
|---|---|---|
| Template cDNA (1:10) | | 2 μl |
| GD F | 10 pM | 0.5 μl |
| GD R | 10 pM | 0.5 μl |
| dNTP's | 10 mM | 0.5 μl |
| Taq DNA polymerase | 3 U/μl | 0.3 μl |
| Taq buffer A | 10X | 3 μl |
| Milli Q water | | 24.2 μl |
| Total volume | | 30 μl |

### PCR Conditions (Eppendorf Machine):

| Steps | Temperature | Time | Cycle |
|---|---|---|---|
| 1 | 94° C. | 3 mins | |
| 2 | 94° C. | 30 secs | |
| 3 | 69° C. | 50 secs | |
| 4 | 72° C. | 1.30 min | Go to step-2 30X |
| 5 | 72° C. | 10 mins | |
| 6 | 10° C. | ∞ | |

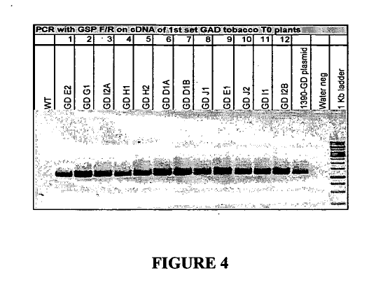

The amplified product was visualized on 0.8% agarose gel as shown in FIG. 4.

### Example 3

### Evidence that Plants with Altered GAD Gene Tolerate Salt Stress at Seedling Stage

Tolerance of the transgenic plants to salt stress was studied in the T1 generation both at seedling stage and during the adult plant stage encompassing the whole life cycle of the plant.

### Salt Tolerance at Seedling Stage on Media

The salt stress experiments were performed with the wild type and T1 GAD transgenic tobacco seedling. The T1 seeds were surface sterilized by washing twice with sterile water (2-3 min) followed by a wash with 70% alcohol for 2 min and then treated with 70% bleach for 10 min and finally washing with sterile water for 5-6 times. The seeds were then blot dried and placed on the ½ MS media plates with different salt concentrations (0, 50 and 200, mM NaCl) and were incubated at 28° C. in the dark for germination. After germination they were shifted to light room under 16 h light and 8 h dark cycle.

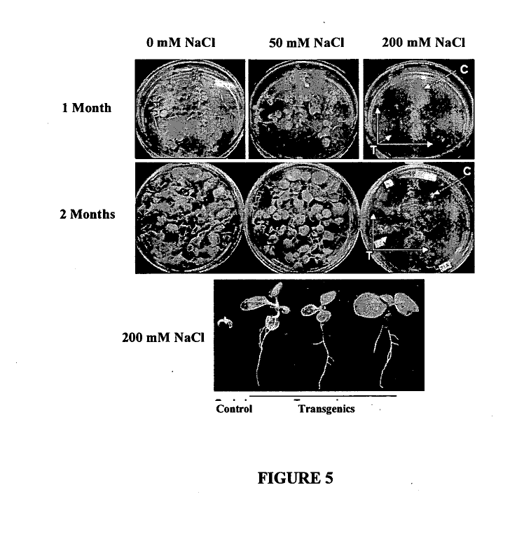

Three of the transgenics events—D1A, E2 and H1 showed tolerance to 200 mM NaCl as compared to the wild type (FIG. 5). The wild type seeds did germinate on 200 mM NaCl but failed to put up a good growth. The presence of high salt concentration in the growth media inhibited the proper growth of the wild type seedlings (plants without the introduced GAD gene) while the presence of high salt did not affect the normal growth of the transgenic seedlings as the introduced GAD gene had rendered them to be tolerant to high salt concentrations in the growth media.

### Salt Tolerance at Seedling Stage Tested on Hydroponics Culture

The two transgenic event E2 and H1 were selected for evaluating the tolerance to high salt and tested in hydroponics culture. The T1 seeds were germinated on moist filter paper discs supplemented with hygromycin (50 mg/L); the positive seedlings that germinated and grew on this were selected and placed on hydroponics floats along with the wild type seedlings.

The hydroponics growth media consisted of 1/10^{th} MS media supplemented with different salt concentrations (100, 200 and 300 mM). The pH in the media was monitored on daily basis and maintained within a range of 5-7. The media was changed once in two days after washing the hydroponics troughs to avoid fungal and algal growth. The final observations were made after five weeks of growth.

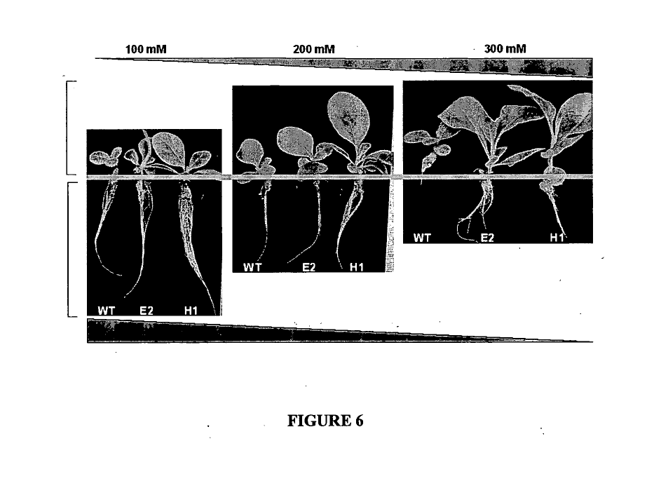

Both the transgenics events—E2 and H1 showed tolerance to 300 mM NaCl as compared to the wild type (FIG. 6). The wild type seeds did germinate and grew on 300 mM NaCl but failed to put up a good growth and were weaker with lesser biomass than the transgenic seedlings. The presence of high salt concentration in the growth media inhibited the proper growth of the wild type seedlings (plants without the introduced GAD gene) while the presence of high salt did not affect the normal growth of the transgenic seedlings as the introduced GAD gene had rendered them to be tolerant to high salt concentrations in the growth media. In this experiment we were able to demonstrate increased salt tolerance of the transgenic plants withstanding salt stress up to 300 mM NaCl.

### Example 4

### Evidence that Plants with Altered GAD Gene Tolerate Salt Stress Throughout their Life Cycle

Tolerance of the transgenic plants to salt stress was studied in the T1 generation during the adult plant stage encompassing the whole life cycle of the plant.

The three transgenic event D1A, E2 and H1 were selected for evaluating the tolerance to high salt and tested in pot culture in the green house. The experiments were performed with the wild type and transgenic tobacco. The T1 seeds were germinated on moist filter paper discs supplemented with hygromycin (50 mg/L); the positive seedlings that germinated and grew on this were selected and placed on soil in big pots (11 inch diameter) along with the wild type seedlings. Seedlings were cultivated in a green house in pots containing mixture of field soil and farmyard manure (FYM). Plants were irrigated with normal water or saline water containing 200 or 300 mM NaCl. The experiments were performed with three treatments and three replications with four genotypes (wild type and D1A, E2 and H1 transgenic tobacco) as indicated in Table 1.

TABLE 1
Experimental design for salt tolerance studies. Three treatments and three
replications were taken for four genotypes for comparison.

| | Treatment-1 (0 mM) | | | | Treatment-2 (200 mM) | | | | Treatment-1 (300 mM) | | | |
|---|---|---|---|---|---|---|---|---|---|---|---|---|
| Replication-1 | WT | D1A | E2 | H1 | WT | D1A | E2 | H1 | WT | D1A | E2 | H1 |
| Replication-2 | WT | D1A | E2 | H1 | WT | D1A | E2 | H1 | WT | D1A | E2 | H1 |
| Replication-3 | WT | D1A | E2 | H1 | WT | D1A | E2 | H1 | WT | D1A | E2 | H1 |

### Phenotypic Evaluation:

The phenotypic characters were observed and parameters like plant height, internodal distance, number of branches, number of leaves, leaf area, stem thickness (girth), total biomass, grain yield etc were recorded.

### Plant Height

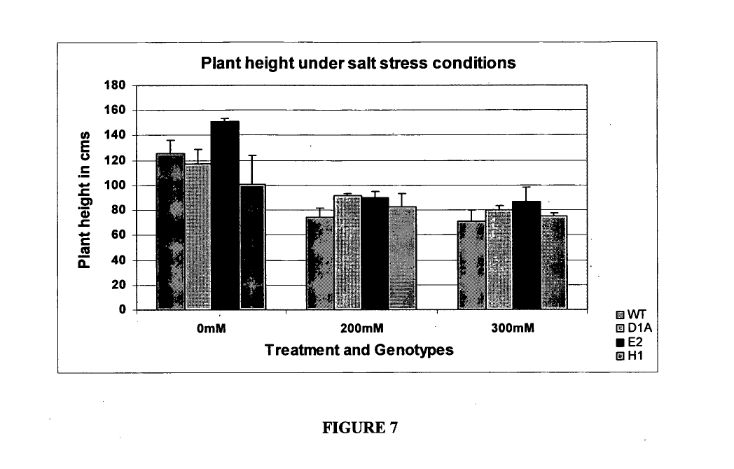

The height of the plant was measured in the transgenic plants and the wild type plants (plants with out the introduced glutamate decarboxylase gene). The plant height was measured using scale from the soil level to the tip of the plant including the inflorescence and the branches. The transgenic showed higher plant height (at least 20% more) during salt stress conditions (200 & 300 mM NaCl) as compared to the wild type plants (FIG. 7).

### Internodal Distance

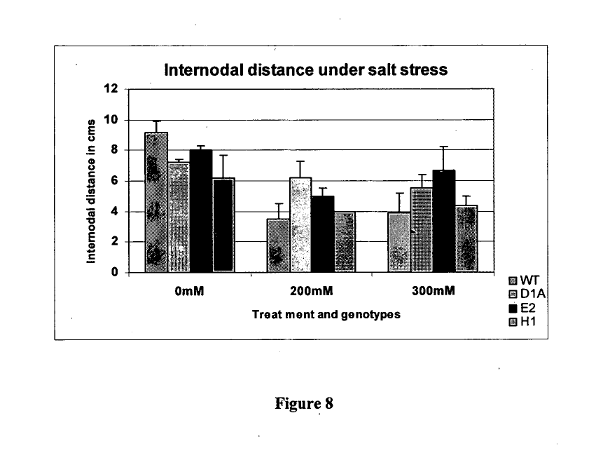

The distance between two internodes on the stem was measured in the transgenic plants and the wild type plants (plants with out the introduced glutamate decarboxylase gene). The internodal distance was measured between the 5^{th} & 6^{th} leaf and 6^{th} & 7^{th} leaf. The leaf was counted from the top with the fully expanded leaf considered to be leaf number-1. The distance was measured using a thread and then measuring the thread length on a scale and expressed in cms. The transgenic showed an increase in internodal distance at higher levels of soil salinity as compared to the wild type (FIG. 8).

### Number of Leaves

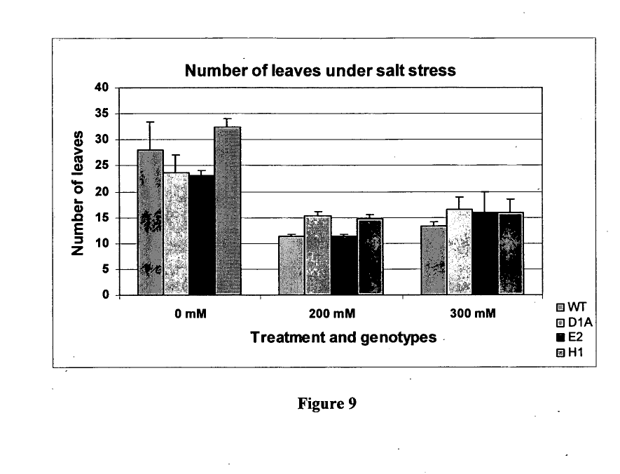

The increase in number of leaves under saline soil conditions (200 & 300 mM NaCl) was observed in the transgenics when compared to wild type (FIG. 9). The transgenics showed at least 20% increase in the leaf number compared to the wild type.

### Stem Girth (Circumference or Stem Thickness)

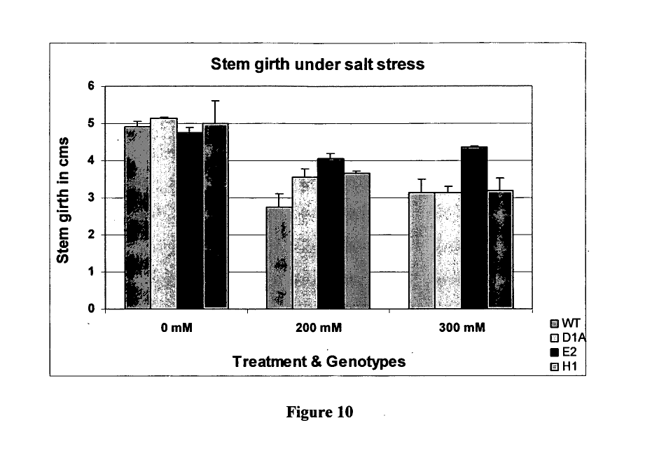

The thickness of the stem was measured in the transgenic plants and the wild type plants (plants with out the introduced glutamate decarboxylase gene). Girth of the stem was measured at a height of 5-6 cms above from the soil level. A thread was used to circle the stem at the appropriate height and then the length of the thread was measured on a scale and expressed in cms. The transgenics showed a thicker stem (27-45% thicker) under 200 mM NaCl conditions compared to the wild type plants (FIG. 10).

### Leaf Area

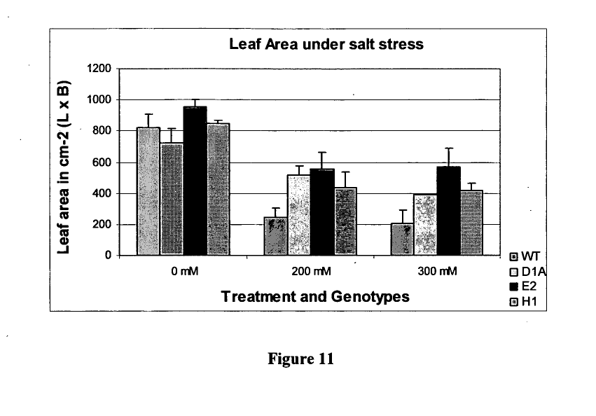

The size of the leaf was measured in the transgenic plants and the wild type plants (plants with out the introduced glutamate decarboxylase gene). The leaf was measured vertically from the node to the tip of the leaf and was considered as the length of the leaf. The breadth of the leaf was measured horizontally at the broadest point and was considered as the breadth of the leaf. The leaf area was calculated as the Length×Breadth expressed in cm⁻² units. Under saline soil conditions (200 & 300 mM NaCl) there was significant increase in the leaf area of the transgenics when compared to the wild type (FIG. 11). The transgenics were observed to have twice the leaf area when compared to the wild type under salt stress conditions.

### Plant Biomass

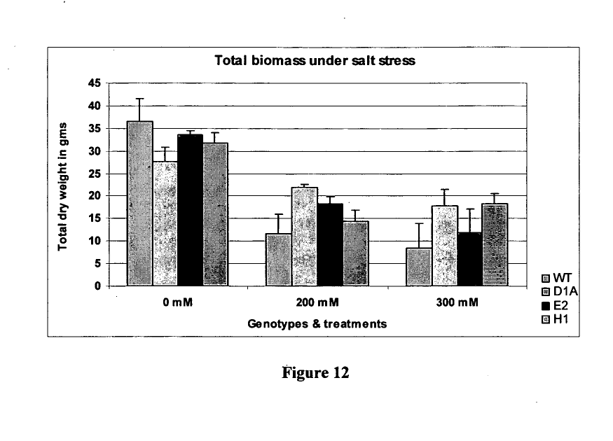

The biomass generated was measured in the transgenic plants and the wild type plants (plants with out the introduced glutamate decarboxylase gene). Plant biomass was estimated as the total plant dry weight. The plant biomass was estimated under different salt stress treatments. The total biomass from the transgenics was significantly higher as compared to the wild types in both 200 and 300 mM NaCl conditions (FIG. 12). The transgenics under salt stress conditions showed at least 30% more biomass than the wild type plants.

### Grain Yield

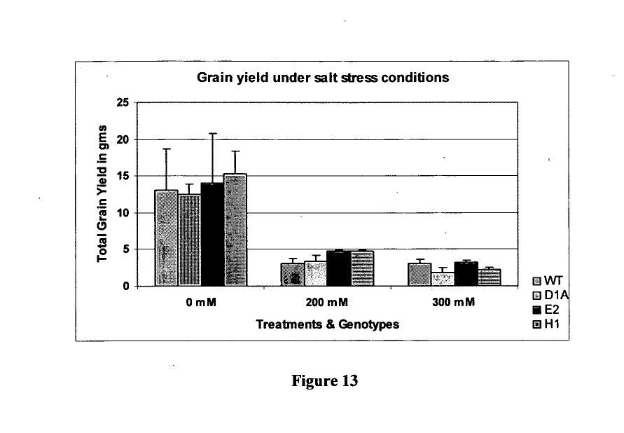

The total grain yield was higher in the transgenics than the wild type under both saline and non-saline conditions (FIG. 13). Although there was reduction in grain yield in the saline conditions when compared to the non-saline conditions, the grain in transgenics was higher compared to the wild type plant under similar conditions.

The GAD transgenics performed better than the wild type plants under high salinity conditions for the different agronomic and physiological status of the plants thus indicating the role of GAD gene for the superior performance of the transgenics under salt stress conditions.

We claim:

1. A method for generating a transformed plant that exhibits enhanced tolerance to environmental stresses, comprising: incorporating into a plant's genome a DNA construct comprising a promoter operably linked to a nucleotide sequence that encodes a functional glutamate decarboxylase (GAD) enzyme.

2. The method according to claim 1, wherein the nucleotide sequence that encodes a functional glutamate decarboxylase enzyme comprises a nucleotide sequence set forth in SEQ ID No. 1.

3. The method according to claim 1, wherein the promoter is selected from the group consisting of a constitutive promoter, an inducible promoter, a tissue specific promoter and a cell type specific promoter operably linked to the nucleotide sequence set forth in SEQ ID No. 1.

4. The method according to claim 3, wherein the promoter selected is from an inducible promoter, responds to a signal selected from the group consisting of mechanical shock, heat, cold, salt, flooding, drought, wounding, anoxia, pathogens, ultraviolet-B, nutritional deprivation, a flowering signal, a fruiting signal, cell specialization and combinations thereof.

5. The method according to claim 3 wherein promoter selected is from tissue specific promoter, expresses in plant tissues selected from the group consisting of leaf, stem, root, flower, petal, anther, ovule etc and combinations thereof.

6. The method according to claim 3 wherein promoter selected is from cell type specific promoter, expresses in plant cells selected from the group consisting of parenchyma, mesophyll, xylem, phloem, guard cell, stomatal cell etc and combinations thereof.

7. The method according to claim 2, wherein the glutamate decarboxylase enzyme comprises an amino acid sequence set forth in SEQ ID NO: 2.

8. The method according to claim 7, wherein the amino acid sequence as set forth in SEQ ID No. 2 is effective to catalyze a reaction of glutamic acid to gamma-amino-butyric acid (GABA).

9. The method according to claim 1, wherein the transformed plant expresses glutamate decarboxylase (GAD) gene set forth in SEQ ID No. 1, at higher level than the level of the GAD gene expressed by a non-transformed plant of the same species under the same conditions.

10. The method according to claim 1, wherein the target plant is selected from the group consisting of monocots, dicots, cereals, forage crops, legumes, pulses, vegetables, fruits, oil seeds, fiber crops, flowers, horticultural, medicinal and aromatic plants.

11. The method of claim 1, wherein said incorporating DNA construct into plant genome comprises;
(i) Transforming a cell, tissue or organ from a host plant with the DNA construct;
(ii) Selecting a transformed cell, cell callus, somatic embryo, or seed which contains the DNA construct;
(iii) Regenerating a whole plant from the selected transformed cell, cell callus, somatic embryo, or seed; and
(iv) Selecting a regenerated whole plant that expresses the polynucleotide.

12. The method according to claim 11 wherein a cell tissue or organ from a host plant is transformed with the DNA construct mediated by using particle gun, biolistic or Agrobacterium.

13. A transformed plant obtained according to claims 1-12 and its progeny thereof.

14. The transformed plant according to claim 13, wherein the DNA construct set forth in SEQ ID No.1 is incorporated into the plant in a heterozygous or homozygous state.

15. The transformed plant according to claims 1-14, wherein the plant exhibits significantly enhanced tolerance to environmental stress selected from the group consisting of salt stress, drought, mechanical shock, heat, cold, salt, flooding, wounding, anoxia, pathogens, ultraviolet-B, nutritional deprivation, and combinations thereof.

16. A plant transformed with a vector comprising a constitutive promoter operably linked to a polynucleotide that encodes a GAD enzyme, or progeny thereof; wherein the plant expresses the polynucleotide; and wherein the plant exhibits significantly improved growth characteristics, yield, reproductive function or other morphological or agronomic characteristic compared to a non-transformed plant.


SEQUENCE LISTING (2 sequences)

SEQ ID NO: 1 - DNA, 1479 bp, Oryza Sativa
  CDS (1)..(1479)
atggtgctctccaaggccgtctccgagagtgacatgtccgtgcactccaccttcgcctcccgctacgtccgcgcctccctcccaaggtaccggatgccggagaactcgatcccgaaggaggcggcgtaccagatcatcaacgacgagctgatgctggacggcaacccgcggctgaacctggcgtcgttcgtcaccacgtggatggagcccgagtgcgacaagctcatcatggccgccatcaacaagaactacgtcgacatggacgagtaccccgtcaccaccgagctccagaaccggtgcgtgaacatgatcgcgcacctgttccacgcgccgctcggggaggacgagacggcggtgggcgtgggcacggtgggttcgtcggaggccatcatgctggccgggctggccttcaagcggcggtggcagaacaagcgcaaggccgaggggaagccgttcgacaagcccaacatcatcaccggcgccaacgtgcaggtgtgctgggagaagttcgcccgctacttcgaggtggagctcaaggaggtgaagctccgcgacggctactacgtcatggaccccgagaaggccgtcgacatggtcaacgagaacaccatctgcgtcgccgccatcctcggctccaccctcaacggcgagttcgaggacgtcaagctactcaacgacctcctcgacaagaagaacaaggagactgggtgggagacgccgatccacgtggacgcggcgagcggcgggttcatcgcgccgttcctgtacccggagctggagtgggacttccggctgccgtgggtgaagagcatcaacgtgagcggtcacaagtacgggctcgtctacgccggcatcggctggtgcatctggcgcaacaaggaggacctgcccgaggagctcatcttccacatcaactacctcggcaccgaccagccaaccttcaccctcaacttctccaagggctccagccaggtcatcgcccagtactaccagctcatccgccacggcttcgaggggtacaggaacatcatggagaactgccacgagaacgcgatggtgctgaaggaagggctggtgaagacggggaggttcgacatcgtgtccaaggacgaaggggtgccgctggtggcgttctcgctcaaggaccggagccggcacgacgagttcgagatctccgacatgctgcgccgcttcggctggatcgtgccggcgtacaccatgccgcccgacgcccagcacgtcacggtgctccgcgtggtcatccgggaggagttcagccgcaccctcgccgagcgcctcgtcctcgacatcgagaaggtgatgtaccagctcgacgcgctcccctccaggctcatgccccccgtgccgccggcgccgctgctggtggtcgccaagaagtcggagctcgagacgcagcggtcggtgacggaggcgtggaagaagttcgtgctcgccaagaggaccaacggcgtctgctag

SEQ ID NO: 2 - protein, 492 aa, Oryza Sativa
MVLSKAVSESDMSVHSTFASRYVRASLPRYRMPENSIPKEAAYQIINDELMLDGNPRLNLASFVTTWMEPECDKLIMAAINKNYVDMDEYPVTTELQNRCVNMIAHLFHAPLGEDETAVGVGTVGSSEAIMLAGLAFKRRWQNKRKAEGKPFDKPNIITGANVQVCWEKFARYFEVELKEVKLRDGYYVMDPEKAVDMVNENTICVAAILGSTLNGEFEDVKLLNDLLDKKNKETGWETPIHVDAASGGFIAPFLYPELEWDFRLPWVKSINVSGHKYGLVYAGIGWCIWRNKEDLPEELIFHINYLGTDQPTFTLNFSKGSSQVIAQYYQLIRHGFEGYRNIMENCHENAMVLKEGLVKTGRFDIVSKDEGVPLVAFSLKDRSRHDEFEISDMLRRFGWIVPAYTMPPDAQHVTVLRVVIREEFSRTLAERLVLDIEKVMYQLDALPSRLMPPVPPAPLLVVAKKSELETQRSVTEAWKKFVLAKRTNGVC